(19) United States
(12) Patent Application Publication
(10) Pub. No.: US 2020/0360607 A1
(43) Pub. Date: Nov. 19, 2020

(54) SYSTEMS AND METHODS FOR PROVIDING PRESCRIBED INFUSION TREATMENTS

(71) Applicant: INFUSYSTEM HOLDINGS, INC., Madison Heights, MI (US)

(72) Inventors: Jean Raniere Pfeiffer, Madison Heights, MI (US); Carrie Ann Lachance, Hooksett, NH (US); David Joseph Bonar, Rossmoor, CA (US)

(21) Appl. No.: 16/413,248

(22) Filed: May 15, 2019

(51) Int. Cl. A61M 5/172; G16H 20/17; G16H 50/30; G16H 40/67

(52) U.S. Cl. CPC A61M 5/172; G16H 20/17; A61M 2205/18; G16H 40/67; G16H 50/30

(57) ABSTRACT

A method for mitigating the risk of providing improper infusion dosages to patients is provided. The method is performed by an infusion safety server which includes a processor, a server memory, and a server database. The method includes receiving an initiation instruction from a clinician computer system to begin a safety check process. The initiation instruction includes a set of prescribed infusion parameters. The method includes instructing a patient computer system to transmit a resolution message including a set of resolution infusion parameters, within a predetermined timeout period. The method includes transmitting a timeout alert to a support computer system if the resolution message is not received within the predetermined timeout period. The method includes transmitting an infusion safety alert to the support computer system if the set of resolution infusion parameters is determined to not match the set of prescribed infusion parameters.

### FIELD OF INVENTION

The field relates to administration of medications through infusion systems and, more specifically, to methods and systems for mitigating the risk of providing excess or inadequate infusion to medical patients receiving infusion treatments.

### BACKGROUND OF THE DISCLOSURE

Infusion systems are increasingly used for the administration of medicine to patients with a variety of conditions. In particular, infusion pumps are used to infuse liquids into the body of a patient. More specifically, infusion pumps are typically applied for intravenous infusion, but they may also be applied for subcutaneous, arterial, epidural infusions, and other types of infusions.

Infusion pumps are especially popular for use in the administration of medicine that is expensive, requires a long period of dosage, or has complicated dosing directions. As such, infusion pumps are especially popular in the administration of, for example, chemotherapy drugs. A key benefit of infusion pumps is that they are capable of administering fluids in a manner that would be expensive or unreliable if it were administered manually by health care providers. For example, infusion pumps can be applied at dosage levels that cannot be effectively performed through an intravenous drip, and can provide precise dosages at exact time intervals, and can accommodate complex administration variables such as varying dosages throughout a day. Additionally, portable infusion pumps can frequently be administered to patients receiving care at home or in outpatient facilities that provide infusion services. Because of this, infusion pump treatments are also significantly less expensive than treatments in hospitals.

Despite these significant benefits, the use of infusion pumps can carry significant risks. Because infusion pumps are frequently used in outpatient infusion suites, or in at-home settings, there may be elevated risks of overmedication or undermedication. The primary concern is ensuring that the infusion of medicine provided by an infusion pump corresponds to the prescription for medicine set by a health care provider. Generally, infusion pumps are themselves reliable, but they require coding or programming by a user who is typically a nurse or another healthcare provider. The step of user coding or programming introduces risks resulting from dose miscalculations and errors made by a user, improper programming of infusion pumps, and a lack of control and checks to ensure that the infusion of medicine provided by a particular pump properly corresponds to a prescribed dosage of that medicine.

The adverse consequences resulting from improper infusion can be serious. Because infusion pumps are frequently used to provide chemotherapy drugs, the consequences of overmedication can be dangerous, or even fatal. In known cases, patients have received overmedication resulting from programming errors of an infusion pump, or misunderstandings of the proper dosage to be administered. In many such cases, the patients experienced toxic side effects and suffered illness or death.

Further, because the medication administered via infusion pumps is often of critical importance to a patient's health, there is a risk associated with providing an improperly low level of medication to a patient via infusion pumps. For the same reasons identified above (i.e., dose miscalculations and errors made by a user, improper programming of infusion pumps, and a lack of control and checks) there is a risk of providing improperly low levels of medication through infusion pumps.

Accordingly, a solution to these technical problems is desired that can provide ensure that the levels of medication provided by an infusion pump corresponds to the prescribed dosage associated with a patient.

### BRIEF SUMMARY OF THE INVENTION

In one aspect, an infusion safety system for mitigating the risk of providing improper infusion dosages to patients is provided. The infusion safety system includes a clinician computer system. The clinician computer system includes a clinician processor and a clinician memory. The clinician memory is encoded with a clinician infusion safety application programmed to run on the clinician processor. The infusion safety system also includes a patient computer system further including a patient processor and a patient memory. The patient memory is encoded with a patient infusion safety application programmed to run on the patient processor. The infusion safety system further includes a support computer system including a support processor and a support memory. The support memory is encoded with a support infusion safety application programmed to run on the support processor. The infusion safety system additionally includes a server. The server includes a server processor, a server memory, and a server database. The server memory includes an engine configured to run on the server processor. The server is in networked communication, via the engine, with the clinician computer system, with the patient computer system, and with the support computer system. The server processor is configured to receive an initiation instruction from the clinician computer system to begin a safety check process. The initiation instruction includes a set of prescribed infusion parameters. The server processor is further configured to instruct the patient computer system to transmit a resolution message including a set of resolution infusion parameters within a predetermined timeout period. The server processor is additionally configured to transmit a timeout alert to the support computer system if the resolution message is not received within the predetermined timeout period. The server processor is also configured to transmit an infusion safety alert to the support computer system if the set of resolution infusion parameters is determined to not match the set of prescribed infusion parameters.

In another aspect, an infusion safety server for mitigating the risk of providing improper infusion dosages to patients is provided. The infusion safety server includes a processor, a server memory, and a server database. The server memory includes an engine configured to run on the processor. The server is in networked communication, via the engine, with a clinician computer system, with a patient computer system, and with a support computer system. The processor is configured to receive an initiation instruction from the clinician computer system to begin a safety check process. The initiation instruction includes a set of prescribed infusion parameters. The processor is further configured to instruct the patient computer system to transmit a resolution message including a set of resolution infusion parameters, within a predetermined timeout period. The processor is also configured to transmit a timeout alert to the support computer system if the resolution message is not received within the predetermined timeout period. The processor is additionally configured to transmit an infusion safety alert to the support computer system if the set of resolution infusion parameters is determined to not match the set of prescribed infusion parameters.

In yet another aspect, a method for mitigating the risk of providing improper infusion dosages to patients is provided. The method is performed by an infusion safety server which includes a processor, a server memory, and a server database. The server memory includes an engine configured to run on the processor. The server is in networked communication, via the engine, with a clinician computer system, with a patient computer system, and with a support computer system. The method includes receiving an initiation instruction from the clinician computer system to begin a safety check process. The initiation instruction includes a set of prescribed infusion parameters. The method also includes instructing the patient computer system to transmit a resolution message including a set of resolution infusion parameters, within a predetermined timeout period. The method further includes transmitting a timeout alert to the support computer system if the resolution message is not received within the predetermined timeout period. The method additionally includes transmitting an infusion safety alert to the support computer system if the set of resolution infusion parameters is determined to not match the set of prescribed infusion parameters.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 illustrates an exemplary configuration of a computer system, as described herein.

FIG. 2 illustrates exemplary infusion safety system, as described herein, using a variety of computer systems as shown in FIG. 1.

FIG. 3 is a flowchart illustrating process carried out by the infusion safety system of FIG. 2.

FIG. 4 is a flow diagram representing a method for mitigating the risk of providing improper infusion dosages to patients, shown from the perspective of the infusion safety server shown in FIG. 2.

FIG. 5 is a diagram of elements of one or more example computer systems that may be used in the system and method shown in FIGS. 3 and 4.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

As required, a detailed embodiment of the present invention is disclosed herein; however, it is to be understood that the disclosed embodiment is merely exemplary of the principles of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

As used herein, an “infusion pump” refers to a medical device that infuses fluids, medication or nutrients into a patient's circulatory system. Infusion pumps are generally used intravenously, although subcutaneous, arterial and epidural infusions are occasionally used. Infusion pumps can administer fluids in ways that would be impractically expensive or unreliable if performed manually by nursing staff.

In view of known problems identified above, there is a risk of providing improperly low levels of medication through infusion pumps.

Accordingly, a solution to these technical problems is desired that can provide ensure that the levels of medication provided by an infusion pump corresponds to the prescribed dosage associated with a patient.

The systems and methods described herein provide a practical application to address the technological and medical problem of improper provision of medication through infusion pumps. In particular, an infusion safety system is described to provide two-way confirmation from both a clinician computer system and a patient computer system to ensure that the infusion provided to a patient corresponds to the infusion prescribed by a clinician. The infusion safety system includes multiple computer systems and is controlled from by an infusion safety server. In one example, the infusion safety system includes the infusion safety server, the patient computer system, the clinician computer system, and a support computer system. In other examples, additional computer systems may be used.

In the example embodiment, the infusion safety server includes a variety of application program interfaces (APIs) to facilitate interaction with the other computer systems. First, the infusion safety server includes an enrollment API for enrolling a patient and patient computer system to use the infusion safety server. Second, the infusion safety server may include a patient consent API for processing and documenting patient consent. Third, the infusion safety server includes a mobile API for coordinating interactions between the clinician computer system, the patient computer system, and the support computer system to perform the methods described herein. These APIs are described in more detail below. As described herein, the clinician computer system, patient computer system, and support computer system are all in networked communication with one another at least through the infusion safety server. Further, the systems and servers described may communicate using any suitable wired or wireless communications protocols including Bluetooth®, any 802.11 standard, Zigbee, or any other communication protocol.

In the example embodiment, in order to interact with the infusion safety server, each of the computer systems may include a client application that is configured to communicate with the infusion safety server directly or via the APIs. Specifically, the patient computer system includes a patient infusion safety application, the clinician computer system includes a clinician infusion safety application, and the support computer system includes a support infusion safety application. Such client applications may be provided to the computer system through any suitable means including via download from an application store or a website.

In the example embodiment, via the patient computer system, a patient accesses an online application to create a new patient profile and enroll in the infusion safety system. Specifically, via the patient computer system, the patient selects an option to create a new patient profile associated with the mobile API. In some examples, a clinician may assist the patient and use the patient computer system to select the option and create the new patient profile, on behalf of the patient. The patient computer system is used to provide enrollment information including, for example, a start date, a telephone number, an email address, an infusion pump product identifier, a catheter care kit identifier, an infusion treatment plan, prescribed infusion parameters including rate and volume, clinician identifiers, language preference, and codes for any diagnoses associated with the patient. After the patient computer system provides such information, the infusion safety server enrolls the patient computer system by recording an entry for that patient computer system in the server database. (After it is provided, the patient computer system may subsequently update or revise such enrollment information. If the patient computer system is used to revise the infusion treatment plan or the prescribed infusion parameters, the online application may require confirmation of such changes to avoid error.) Further, the infusion safety server generates and provides a link to the patient computer system. The patient computer system uses the link to access the patient infusion safety application via any suitable application provider. The infusion safety server also generates an access code specific to the patient computer system. In use, the access code is used to authenticate the patient computer system and secure patient information.

In one example embodiment, prior to using the infusion safety system, the patient may provide consent information through the patient consent API. In some embodiments, the infusion safety system may be used by a patient without providing consent. Specifically, the patient infusion safety application provides a mechanism for tracking patient consent and signed paperwork using the patient consent API. In one example embodiment, once consent forms are provided to the patient consent API, the patient computer system may utilize the infusion safety system. Further, consent records and logs associated with the patient computer system are recorded at the server database in a database log of consents and a document repository containing signed consent forms.

In the example embodiment, via the clinician computer system, a clinician also accesses an online application to create a new clinician profile and enroll in the infusion safety system. Specifically, via the clinician computer system, the clinician selects to create a new clinician profile associated with the mobile API. The clinician computer system is used to provide enrollment information including, for example, clinician name, clinician contact information, clinician address, and facility name. After the clinician computer system provides such information, the infusion safety server enrolls the clinician computer system by recording an entry for that clinician computer system in the server database. After the clinician computer system is enrolled, the clinician computer system may access the clinician infusion safety application via any suitable application provider.

Similarly, a support infusion safety application may be downloaded to the support computer system that may be used to interact with the mobile API.

In the example embodiment, when the patient infusion safety application is installed on the patient computer system, a user may initiate the use of the infusion safety system for their infusion by selecting an “activate” option from the patient infusion safety application. In one example, the user is a patient, and in another it is a clinician assisting the patient. In practice, selection of the “activate” option indicates that the user intends to begin infusion for the patient according to a prescribed protocol. At this point, the infusion safety system begins measures to ensure that the infusion proceeds according to the prescribed protocol.

When the patient makes an “activate” selection, the patient infusion safety application transmits an initiation instruction to the mobile API on the infusion safety server. Upon receiving such an initiation instruction, the infusion safety server generates a confirmation message based on the patient record. The patient record is the record that was created in the server database when the patient computer system transmitted a registration request to the mobile API and provided profile information including a set of prescribed infusion parameters and a patient profile. Accordingly, the confirmation message is generated in part based on the set of prescribed infusion parameters, and represents a request for the clinician computer system to confirm the set of prescribed infusion parameters. The infusion safety server transmits the generated confirmation message to the clinician computer system which presents it within the clinician infusion safety application. The confirmation message requests that the clinician authenticate themselves with an access code, and confirm that the prescribed infusion parameters are correct. If the clinician declines to confirm the prescribed infusion parameters, the clinician infusion safety application transmits this selection to the mobile API which transmits a message to the patient infusion safety application indicating an alert or error in the infusion protocol. In such examples, the mobile API may also send a message to the patient infusion safety application indicating that the patient should contact the clinician, or vice versa. If the clinician confirms the prescribed infusion parameters, the clinician infusion safety application transmits this selection to the mobile API which begins a countdown of a coordinated timer, or a safety timer. At the same time, the mobile API instructs the patient computer system to transmit a resolution message including a set of resolution infusion parameters, within a predetermined timeout period.

In the example embodiment, the server database contains an alert protocol defining the predetermined timeout period. Specifically, the predetermined timeout period indicates the length of time that the patient has to create and transmit the resolution message to the mobile API. The patient computer system can respond at any point within the predetermined timeout period. The alert protocol may also define a set of warnings that may be provided prior to the conclusion of the predetermined timeout period. In one example, the alert protocol is defined such that the patient computer system receives an alert requesting the resolution message after a first wait time of, for example, forty-five minutes. In this example, the alert protocol may further define that the patient receives three additional warnings after the next consecutive periods of five minutes. In this example, after the three additional warnings have concluded (i.e., after fifteen minutes of warnings and one hour in total elapsed time), the alert protocol defines that the predetermined timeout period is concluded. Accordingly, the server database is configured to retrieve the alert protocol and define the predetermined timeout period used in the instruction to the patient computer system based on the alert protocol. Similarly, the server database is configured to transmit a warning alert to the patient computer system if the resolution message is not received within a predetermined warning period as defined by the alert protocol. For example, the server database will transmit warnings if the patient computer system fails to provide the resolution message after each of forty-five, fifty, and fifty-five minutes.

In the example embodiment, when the infusion safety server instructs the patient computer system to transmit a resolution message, this represents a request for the patient computer system to send a resolution value (indicating a rate and/or volume of infusion) from the infusion pump associated with the patient. In some examples, this resolution value is displayed on the screen of the infusion pump. In some examples, the resolution value may be provided directly from an infusion pump that is in communication or otherwise integrated with the patient computer system. In some such examples, the resolution value may be automatically provided. In all embodiments, the resolution value may be provided by the patient computer system to the infusion safety server at any point within the predetermined warning period.

If the resolution message is not received prior to the predetermined warning period, as defined above, the infusion safety server is configured to transmit a warning to the patient infusion safety application for display on the patient computer system, wherein the warning advises the patient to provide the resolution message. If the resolution message is not received prior to the predetermined timeout period, the infusion safety server transmits a timeout alert to the support computer system. In one example, transmitting the timeout alert represents sending an electronic communication such as an electronic mail, instant message, text message, or other suitable communication, to the support computer system operated by, for example a nurse or another clinician. The timeout alert provides the support computer system with suitable information regarding the patient from the server database, and the timeout. The nurse or clinician then calls or otherwise contacts the patient. In at least some examples, if the resolution message is not received prior to the predetermined timeout period, the infusion safety server also transmits an alert to the patient infusion safety application for display on the patient computer system, wherein the alert advises the patient to contact a nurse or a clinician for assistance.

If the patient computer system transmits the resolution message before the expiry of the predetermined timeout period, the infusion safety server is also configured to compare the set of resolution infusion parameters in the resolution message (i.e., identified rate and/or volume) to the set of prescribed infusion parameters confirmed by a clinician (i.e., the prescribed rate and/or volume). Further, the infusion safety server determines if the set of resolution infusion parameters fails to match the set of prescribed infusion parameters and, if so, transmits an infusion safety alert to the support computer system. In one example, transmitting the infusion safety alert represents sending an electronic communication such as an electronic mail, instant message, text message, or other suitable communication, to the support computer system operated by, for example a nurse or another clinician. The infusion safety alert includes the set of resolution infusion parameters and the set of prescribed infusion parameters, and includes suitable information regarding the patient that is recorded in the server database. The nurse or clinician then calls or otherwise contacts the patient.

In the example embodiment, the infusion safety server identifies a set of boundary limits associated with the set of prescribed infusion parameters. The infusion safety server also determines if the resolution infusion parameters match the set of prescribed infusion parameters by determining if the set of resolution infusion parameters is within the boundary limits.

In one example, the infusion safety server identifies the set of boundary limits based upon input from a clinician computer system or system defaults. In other examples, the infusion safety server retrieves such information from the server database that was provided by the patient computer system or clinician computer system during registration and included in the server database. In a further example, the infusion safety server identifies an infusion category associated with the patient computer system by, for example, identifying the infusion pump or infusion protocol associated with the patient computer system. In this example, the infusion safety server also retrieves an infusion boundary definition associated with the infusion category from the server database, and identifies the set of boundary limits by processing the infusion boundary definition and the set of prescribed infusion parameters.

A technical effect of the systems and methods described herein is achieved by performing at least one of the following steps: (a) receiving an initiation instruction from the clinician computer system to begin a safety check process, wherein the initiation instruction includes a set of prescribed infusion parameters; (b) instructing the patient computer system to transmit a resolution message including a set of resolution infusion parameters, within a predetermined timeout period; (c) transmitting a timeout alert to the support computer system if the resolution message is not received within the predetermined timeout period; (d) transmitting an infusion safety alert to the support computer system if the set of resolution infusion parameters is determined to not match the set of prescribed infusion parameters; (e) retrieving an alert protocol from the server database; (f) defining the predetermined timeout period used in the instruction to the patient computer system based on the alert protocol; (h) identifying a set of boundary limits associated with the set of prescribed infusion parameters; (i) determining if the resolution infusion parameters match the set of prescribed infusion parameters by determining if the set of resolution infusion parameters is within the boundary limits; (j) identifying an infusion category associated with the patient computer system; (k) retrieving an infusion boundary definition associated with the infusion category from the server database; (l) identifying the set of boundary limits by processing the infusion boundary definition and the set of prescribed infusion parameters; (m) receiving a registration request including a set of prescribed infusion parameters and a patient profile, from the patient computer system; (n) registering a patient record the patient computer system in the server database based on the registration request; (o) receiving an initiation instruction from the patient computer system; (p) generating a confirmation message based on the patient record, wherein the confirmation message requests the clinician computer system confirm the set of prescribed infusion parameters; (q) receiving the initiation instruction generated by the clinician computer system in response to the confirmation message; and (r) transmitting a warning alert to the patient computer system if the resolution message is not received within a predetermined warning period.

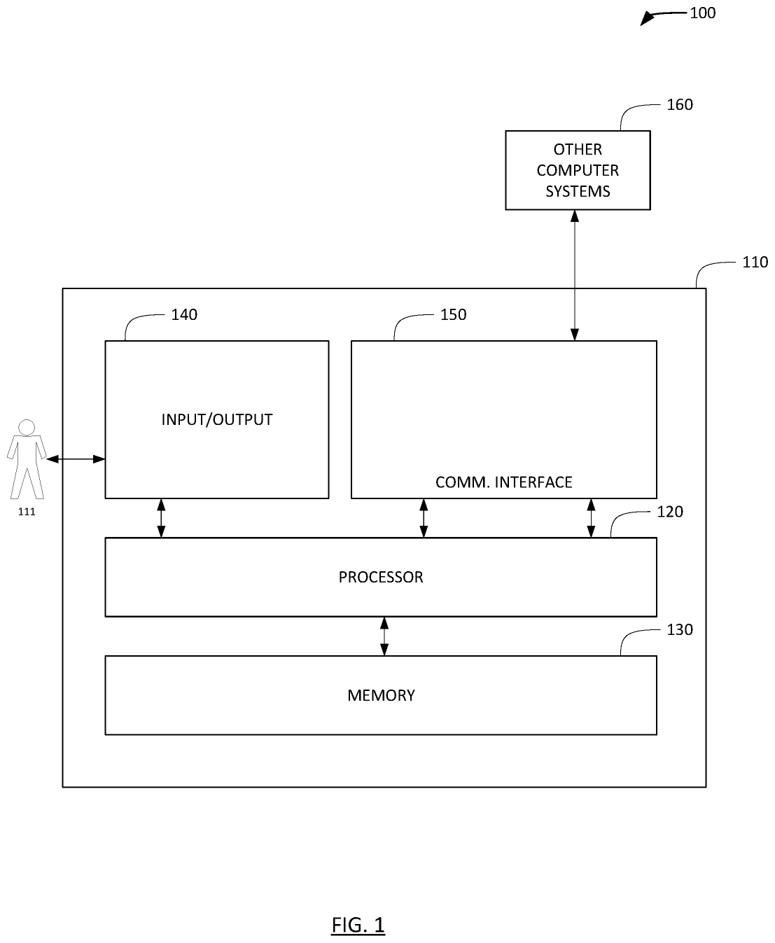

FIG. 1 illustrates an exemplary configuration 100 of a computer system as described herein such as clinician computer system, patient computer system, support computer system, or infusion safety server. Specifically, FIG. 1 illustrates an exemplary configuration 100 of a computer system 110 operated by a user 111 in accordance with one embodiment of the present invention. Computer system 110 may include, but is not limited to, computer systems built into infusion pumps, mobile computer systems, stationary computer systems, computing peripheral devices, smart phones, wearable computer systems, medical computer systems, and vehicular computer systems. Alternatively, computer system 110 may be any computer system capable of performing the infusion safety methods described herein. In some variations, the characteristics of the described components may be more or less advanced, primitive, or non-functional.

In the exemplary embodiment, computer system 110 includes a processor 120 for executing instructions. In some embodiments, executable instructions are stored in a memory area 130. Processor 120 may include one or more processing units, for example, a multi-core configuration. Memory area 130 is any device allowing information such as executable instructions and/or written works to be stored and retrieved. Memory area 130 may include one or more computer readable media.

Computer system 110 also includes at least one input/output component 140 for receiving information from and providing information to user 111. In some examples, input/output component 140 may be of limited functionality or non-functional as in the case of some wearable computer systems. In other examples, input/output component 140 is any component capable of conveying information to or receiving information from user 111. In some embodiments, input/output component 140 includes an output adapter such as a video adapter and/or an audio adapter. Input/output component 140 may alternatively include an output device such as a display device, a liquid crystal display (LCD), organic light emitting diode (OLED) display, or “electronic ink” display, or an audio output device, a speaker or headphones. Input/output component 140 may also include any devices, modules, or structures for receiving input from user 111. Input/output component 140 may therefore include, for example, a keyboard, a pointing device, a mouse, a stylus, a touch sensitive panel, a touch pad, a touch screen, a gyroscope, an accelerometer, a position detector, or an audio input device. A single component such as a touch screen may function as both an output and input device of input/output component 140. Input/output component 140 may further include multiple sub-components for carrying out input and output functions.

Computer system 110 may also include a communications interface 150, which may be communicatively coupleable to a remote device such as a remote computer system, a remote server, or any other suitable system. Communication interface 150 may include, for example, a wired or wireless network adapter or a wireless data transceiver for use with a mobile phone network, Global System for Mobile communications (GSM), 3G, 4G, or other mobile data network or Worldwide Interoperability for Microwave Access (WIMAX). Communications interface 150 is configured to allow computer system 110 to interface with any other computer system using an appropriate wireless or wired communications protocol such as, without limitation, BLUETOOTH®, Ethernet, or IEE 802.11. Communications interface 150 allows computing device 110 to communicate with any other computer systems 160.

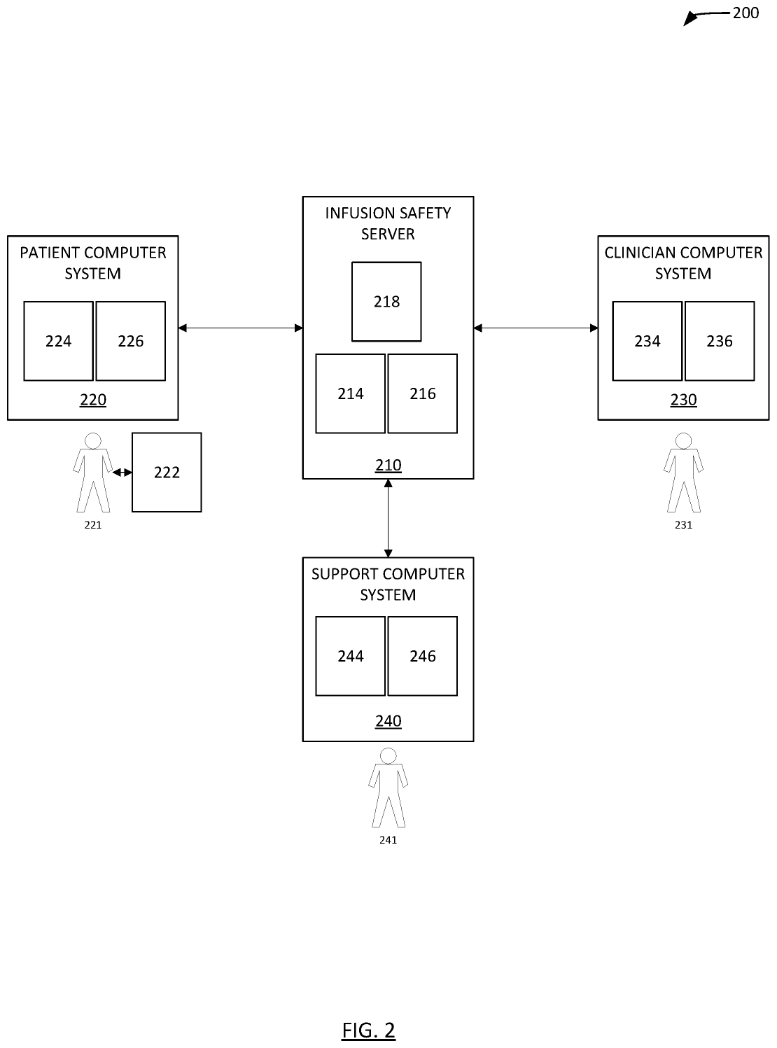

FIG. 2 illustrates exemplary infusion safety system 200, as described herein, using a variety of computer systems as shown in FIG. 1, which are presented in simplified form in this view. In the example embodiment, infusion safety system 200 includes infusion safety server 210 further including processor 214, memory 216, and database 218. Memory 216 is encoded with executables for applications, including an engine, that are run on processor 214 and which can provide the functionalities described herein including, but not limited to, providing the application program interfaces (APIs) described herein and, more specifically, the enrollment API, mobile API, and consent API. In some examples, these features may be combined into fewer components. Database 218 is designed to include information described herein including patient registration (or enrollment) information, clinician registration information, support registration information, consent log information, and electronic records. Infusion safety system 200 also includes patient computer system 220 associated with patient 221. In the example embodiment, patient 221 is receiving an infusion treatment from infusion pump 222 which is programmable. In the example embodiment, infusion pump 222 is configured to provide infusion to patient 221 and to display information associated with the infusion including flow rate and volume. In some examples, infusion pump 222 is in communication with patient computer system 220 and may automatically provide such information to patient computer system 220. In the example embodiment, patient computer system 220 is able to access the Internet to access the online application at which a patient can register for the infusion safety system 200. Patient computer system 220 also includes processor 224 and memory 226, and may be configured to encode a patient infusion safety application on memory 226 for execution by processor 224.

Infusion safety system 200 also includes clinician computer system 230 associated with clinician 231. In the example embodiment, clinician computer system 230 is able to access the Internet to access the online application at which a clinician can register for the infusion safety system 200. Clinician computer system 230 also includes processor 234 and memory 236, and may be configured to encode a clinician infusion safety application on memory 236 for execution by processor 234.

Infusion safety system 200 also includes support computer system 240 associated with support staff 241. In the example embodiment, support computer system 240 is able to access the Internet to access the online application at which a support staff can register for the infusion safety system 200. Support computer system 240 also includes processor 244 and memory 246, and may be configured to encode a support infusion safety application on memory 246 for execution by processor 244.

Using any suitable networking or communications protocol, computer systems 210, 220, 230, and 240 may communicate through wired or wireless communication. In the example embodiment, computer systems 220, 230, and 240 are configured to interact with infusion safety server 210 through at least their respective safety applications interacting with the API running on the engine of the infusion safety server.

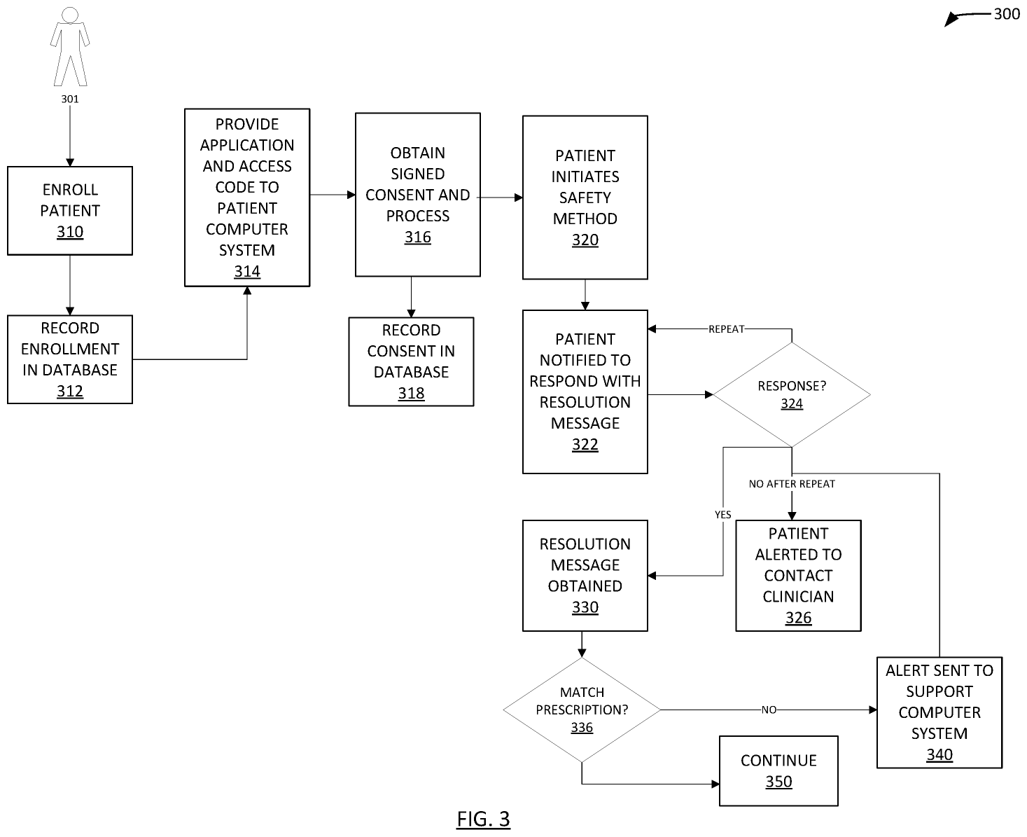

FIG. 3 is a flowchart 300 illustrating process carried out by the infusion safety system 200 of FIG. 2 from the perspective of the patient computer system. Specifically, flowchart 300 illustrates example aspects of the methods described herein from the perspective of the patient computer system. Initially, user 301 begins enrolling a patient computer system with the infusion safety system described. In the example embodiment, user 301 is a clinician associated with a hospital or facility. In other embodiments, user 301 may be any user capable of performing these steps. User 301 enrolls 310 a patient by completing an online application and providing infusion safety server with information including, for example, a start date, a telephone number, an email address, an infusion pump product identifier, a catheter care kit identifier, an infusion treatment plan, prescribed infusion parameters including rate and volume, clinician identifiers, language preference, and codes for any diagnoses associated with the patient. After user 301 provides such information via patient computer system, the infusion safety server enrolls the patient computer system by recording an entry 312 for that patient computer system in the server database. The infusion safety server generates and provides a link and access code 314 to the patient computer system. The patient computer system uses the link to access the patient infusion safety application via any suitable application provider.

Prior to using the infusion safety system, the patient may provide consent information through the patient consent API. Specifically, the patient infusion safety application provides a mechanism for tracking patient consent and signed paperwork using the patient consent API. In one example embodiment, when consent forms are provided 316 to the patient consent API, the patient computer system may utilize the infusion safety system. Further, consent records and logs associated with the patient computer system may be recorded 318 at the server database in a database log of consents and a document repository containing signed consent forms.

Patient computer system initiates the safety method 320 by selecting an initiation option from patient infusion safety application. The patient computer system is notified 322 to respond with a resolution message (including flow rate and/or volume) wherein such notification displays on patient infusion safety application. The infusion safety server waits for a response 324 based on the alert protocol, and repeats warnings based on that same protocol. The patient computer system can respond with a resolution message at any time during the infusion, and is not required to wait any period of time before responding to the notification 322. If no response is received after waiting for the predetermined timeout period, the patient is alerted 326 to contact a clinician and the infusion safety server sends an alert 340 to the support computer system. If a resolution message is obtained 330, the infusion safety server performs a comparison to determine whether the resolution message contains resolution infusion parameters that match 336 prescribed infusion parameters. If the parameters do not match 336, the infusion safety server sends alert 340 to the support computer system and sends an alert to the patient to contact a clinician. If the parameters match 336, the infusion continues 350.

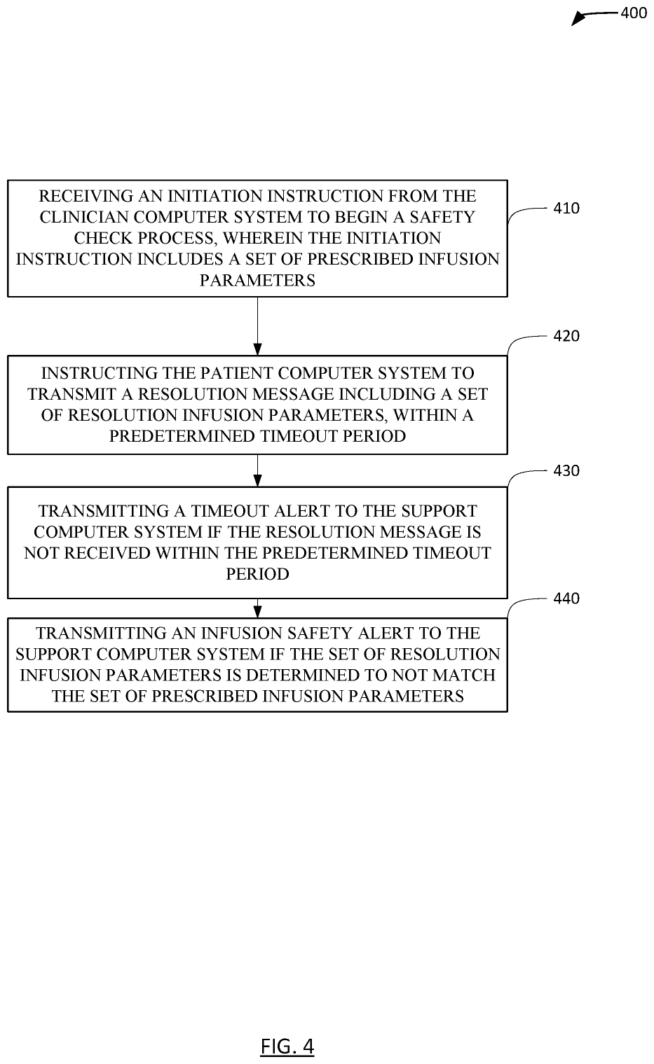

FIG. 4 is a flow diagram representing a method 400 for mitigating the risk of providing improper infusion dosages to patients, shown from the perspective of the infusion safety server shown in FIG. 2. In the example embodiment, method 400 is performed by infusion safety server 210 (shown in FIG. 2). Method 400 includes receiving 410 an initiation instruction from the clinician computer system to begin a safety check process. The initiation instruction includes a set of prescribed infusion parameters. Method 400 also includes instructing 420 the patient computer system to transmit a resolution message including a set of resolution infusion parameters, within a predetermined timeout period. The patient computer system may transmit a resolution message in response at any point within the predetermined timeout period. Method 400 further includes transmitting 430 a timeout alert to the support computer system if the resolution message is not received within the predetermined timeout period. Method 400 additionally includes transmitting 440 an infusion safety alert to the support computer system if the set of resolution infusion parameters is determined to not match the set of prescribed infusion parameters.

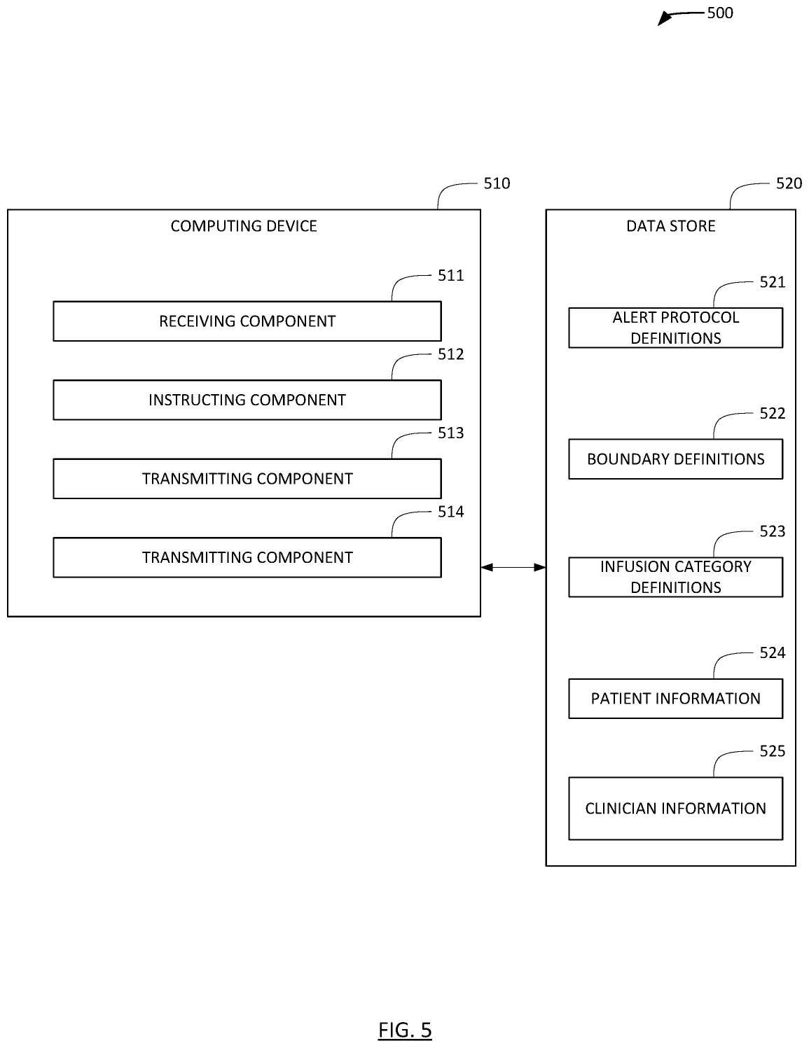

FIG. 5 is a diagram of elements of one or more example computer systems that may be used in the system and method shown in FIGS. 3 and 4. In some embodiments, computing device 510 is similar to computing device 110, 210, 220, 230, or 240, as shown in FIGS. 1 and 2. Data store 520 may be stored at a memory such as memory 130 (shown in FIG. 1) or any other suitable location. Data store 520 may be coupled with several separate components 511, 512, 513, or 514, within computing device 510, which perform specific tasks.

In this embodiment, data store 520 includes alert protocol definitions 521, boundary definitions 522, infusion category definitions 523, patient information 524, and clinician information 525. Computing device 510 may include data store 520, as well as data storage devices (not shown).

Computing device 510 also includes a receiving component 511 for receiving an initiation instruction from the clinician computer system to begin a safety check process, wherein the initiation instruction includes a set of prescribed infusion parameters, an instructing component 512 for instructing the patient computer system to transmit a resolution message including a set of resolution infusion parameters, within a predetermined timeout period, a transmitting component 513 for transmitting a timeout alert to the support computer system if the resolution message is not received within the predetermined timeout period, and a transmitting component 514 for transmitting an infusion safety alert to the support computer system if the set of resolution infusion parameters is determined to not match the set of prescribed infusion parameters.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave). The term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs used in creating the programs and software described may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, Javascript®, HTMLS (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. An infusion safety system for mitigating the risk of providing improper infusion dosages to patients, comprising:
a clinician computer system including a clinician processor and a clinician memory, wherein the clinician memory is encoded with a clinician infusion safety application programmed to run on the clinician processor;
a patient computer system including a patient processor and a patient memory, wherein the patient memory is encoded with a patient infusion safety application programmed to run on the patient processor;
a support computer system including a support processor and a support memory, wherein the support memory is encoded with a support infusion safety application programmed to run on the support processor;
a server comprising a server processor, a server memory, and a server database, wherein the server is in networked communication with the clinician computer system, with the patient computer system, and with the support computer system, the server processor is configured to:
receive an initiation instruction from the clinician computer system to begin a safety check process, wherein the initiation instruction includes a set of prescribed infusion parameters;
instruct the patient computer system to transmit a resolution message including a set of resolution infusion parameters, within a predetermined timeout period;
transmit a timeout alert to the support computer system if the resolution message is not received within the predetermined timeout period; and
transmit an infusion safety alert to the support computer system if the set of resolution infusion parameters is determined to not match the set of prescribed infusion parameters.

2. The infusion safety system of claim 1, wherein the server processor is further configured to:
retrieve an alert protocol from the server database; and
define the predetermined timeout period used in the instruction to the patient computer system based on the alert protocol.

3. The infusion safety system of claim 1, wherein the server processor is further configured to:
identify a set of boundary limits associated with the set of prescribed infusion parameters; and
determine if the resolution infusion parameters match the set of prescribed infusion parameters by determining if the set of resolution infusion parameters is within the boundary limits.

4. The infusion safety system of claim 3, wherein the server processor is further configured to:
identify an infusion category associated with the patient computer system;
retrieve an infusion boundary definition associated with the infusion category from the server database; and
identify the set of boundary limits by processing the infusion boundary definition and the set of prescribed infusion parameters.

5. The infusion safety system of claim 1, wherein the server processor is further configured to:
receive a registration request including a set of prescribed infusion parameters and a patient profile, from the patient computer system; and
register a patient record the patient computer system in the server database based on the registration request.

6. The infusion safety system of claim 5, wherein the server processor is further configured to:
receive an initiation instruction from the patient computer system;
generate a confirmation message based on the patient record, wherein the confirmation message requests the clinician computer system confirm the set of prescribed infusion parameters; and
receive the initiation instruction generated by the clinician computer system in response to the confirmation message.

7. The infusion safety system of claim 1, wherein the server processor is further configured to:
transmit a warning alert to the patient computer system if the resolution message is not received within a predetermined warning period.

8. An infusion safety server for mitigating the risk of providing improper infusion dosages to patients, the infusion safety server comprising a processor, a server memory, and a server database, wherein the server is in networked communication with a clinician computer system, with a patient computer system, and with a support computer system, the processor is configured to:
receive an initiation instruction from the clinician computer system to begin a safety check process, wherein the initiation instruction includes a set of prescribed infusion parameters;
instruct the patient computer system to transmit a resolution message including a set of resolution infusion parameters, within a predetermined timeout period;
transmit a timeout alert to the support computer system if the resolution message is not received within the predetermined timeout period; and
transmit an infusion safety alert to the support computer system if the set of resolution infusion parameters is determined to not match the set of prescribed infusion parameters.

9. The infusion safety server of claim 8, wherein the processor is further configured to:
retrieve an alert protocol from the server database; and
define the predetermined timeout period used in the instruction to the patient computer system based on the alert protocol.

10. The infusion safety server of claim 8, wherein the processor is further configured to:
identify a set of boundary limits associated with the set of prescribed infusion parameters; and
determine if the resolution infusion parameters match the set of prescribed infusion parameters by determining if the set of resolution infusion parameters is within the boundary limits.

11. The infusion safety server of claim 10, wherein the processor is further configured to:
identify an infusion category associated with the patient computer system;
retrieve an infusion boundary definition associated with the infusion category from the server database; and
identify the set of boundary limits by processing the infusion boundary definition and the set of prescribed infusion parameters.

12. The infusion safety server of claim 8, wherein the processor is further configured to:
receive a registration request including a set of prescribed infusion parameters and a patient profile, from the patient computer system; and
register a patient record the patient computer system in the server database based on the registration request.

13. The infusion safety server of claim 12, wherein the processor is further configured to:
receive an initiation instruction from the patient computer system;
generate a confirmation message based on the patient record, wherein the confirmation message requests the clinician computer system confirm the set of prescribed infusion parameters; and
receive the initiation instruction generated by the clinician computer system in response to the confirmation message.

14. The infusion safety server of claim 8, wherein the processor is further configured to:
transmit a warning alert to the patient computer system if the resolution message is not received within a predetermined warning period.

15. A method for mitigating the risk of providing improper infusion dosages to patients, the method performed by an infusion safety server, the infusion safety server comprising a processor, a server memory, and a server database, wherein the server is in networked communication with a clinician computer system, with a patient computer system, and with a support computer system, the method comprising:
receiving an initiation instruction from the clinician computer system to begin a safety check process, wherein the initiation instruction includes a set of prescribed infusion parameters;
instructing the patient computer system to transmit a resolution message including a set of resolution infusion parameters, within a predetermined timeout period;
transmitting a timeout alert to the support computer system if the resolution message is not received within the predetermined timeout period; and
transmitting an infusion safety alert to the support computer system if the set of resolution infusion parameters is determined to not match the set of prescribed infusion parameters.

16. The method of claim 15, further comprising:
retrieving an alert protocol from the server database; and
defining the predetermined timeout period used in the instruction to the patient computer system based on the alert protocol.

17. The method of claim 15, further comprising:
identifying a set of boundary limits associated with the set of prescribed infusion parameters; and
determining if the resolution infusion parameters match the set of prescribed infusion parameters by determining if the set of resolution infusion parameters is within the boundary limits.

18. The method of claim 17, further comprising:
identifying an infusion category associated with the patient computer system;
retrieving an infusion boundary definition associated with the infusion category from the server database; and
identifying the set of boundary limits by processing the infusion boundary definition and the set of prescribed infusion parameters.

19. The method of claim 15, further comprising:
receiving a registration request including a set of prescribed infusion parameters and a patient profile, from the patient computer system; and
registering a patient record the patient computer system in the server database based on the registration request.

20. The method of claim 19, further comprising:
receiving an initiation instruction from the patient computer system;
generating a confirmation message based on the patient record, wherein the confirmation message requests the clinician computer system confirm the set of prescribed infusion parameters; and
receiving the initiation instruction generated by the clinician computer system in response to the confirmation message.
